(19) United States
(12) Patent Application Publication
(10) Pub. No.: US 2009/0113335 A1
(43) Pub. Date: Apr. 30, 2009

(54) DIALYSIS SYSTEM USER INTERFACE

(75) Inventors: Gregory A. Sandoe, Clearwater, FL (US); Peter A. Hopping, Lutz, FL (US); Andres Alvarez-Melendez, Clearwater, FL (US)

Correspondence Address: BAXTER HEALTHCARE CORPORATION, 1 BAXTER PARKWAY, DF2-2E, DEERFIELD, IL, 60015, US

(73) Assignees: Baxter International Inc., Deerfield, IL (US); Baxter Healthcare S.A., Zurich (CH)

(21) Appl. No.: 12/260,654

(22) Filed: Oct. 29, 2008

Related U.S. Application Data
(60) Provisional application No. 60/983,835, filed on Oct. 30, 2007.

(51) Int. Cl. A61M 1/16; A61M 1/28; G06F 3/048

(52) U.S. Cl. 715/773; 604/6.11; 604/29

(57) ABSTRACT

A dialysis system includes a dialysis instrument including a pump actuator; a disposable dialysis fluid carrying set including a pump chamber operable with the pump actuator; and a graphical user interface including a selection highlighting input device and a selection entering input device, the selection highlighting input device configured to enable a user to perform each of: (i) screen navigation selection; (ii) parameter selection including at least one parameter related to the pump actuator and pump chamber; and (iii) value selection including at least one value related to the pump actuator and pump chamber.

### PRIORITY

This application claims priority to and the benefit of U.S. Provisional Patent Application No. 60/983,835, entitled, “Dialysis System User Interface”, filed Oct. 30, 2007.

### BACKGROUND

The examples discussed below relate generally to medical fluid delivery. More particularly, the examples disclose systems, methods and apparatuses for dialysis such as hemodialysis (“HD”) and automated peritoneal dialysis (“APD”).

Due to various causes, a person's renal system can fail. Renal failure produces several physiological derangements. It is no longer possible to balance water and minerals or to excrete daily metabolic load. Toxic end products of nitrogen metabolism (urea, creatinine, uric acid, and others) can accumulate in blood and tissue.

Kidney failure and reduced kidney function have been treated with dialysis. Dialysis removes waste, toxins and excess water from the body that normal functioning kidneys would otherwise remove. Dialysis treatment for replacement of kidney functions is critical to many people because the treatment is life saving.

One type of kidney failure therapy is peritoneal dialysis (“PD”), which infuses a dialysis solution, also called dialysate, into a patient's peritoneal cavity via a catheter. The dialysate contacts the peritoneal membrane of the peritoneal cavity. Waste, toxins and excess water pass from the patient's bloodstream, through the peritoneal membrane and into the dialysate due to diffusion and osmosis, i.e., an osmotic gradient occurs across the membrane. Osmotic agent in dialysis provides the osmotic gradient. The spent dialysate is drained from the patient, removing waste, toxins and excess water from the patient. This cycle is repeated.

There are various types of peritoneal dialysis therapies, including continuous ambulatory peritoneal dialysis (“CAPD”), automated peritoneal dialysis (“APD”), tidal flow dialysate and continuous flow peritoneal dialysis (“CFPD”). CAPD is a manual dialysis treatment. Here, the patient manually connects an implanted catheter to a drain to allow spent dialysate fluid to drain from the peritoneal cavity. The patient then connects the catheter to a bag of fresh dialysate to infuse fresh dialysate through the catheter and into the patient. The patient disconnects the catheter from the fresh dialysate bag and allows the dialysate to dwell within the peritoneal cavity, wherein the transfer of waste, toxins and excess water takes place. After a dwell period, the patient repeats the manual dialysis procedure, for example, four times per day, each treatment lasting about an hour. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysate and to a fluid drain. APD machines pump fresh dialysate from a dialysate source, through the catheter and into the patient's peritoneal cavity. APD machines also allow for the dialysate to dwell within the cavity and for the transfer of waste, toxins and excess water to take place. The source can include multiple sterile dialysate solution bags.

APD machines pump spent dialysate from the peritoneal cavity, though the catheter, and to the drain. As with the manual process, several drain, fill and dwell cycles occur during dialysis. A “last fill” occurs at the end of APD and remains in the peritoneal cavity of the patient until the next treatment.

While APD has obvious lifestyle benefits over CAPD, there is still a need to make dialysis treatments easier to setup and perform.

### SUMMARY

The present disclosure provides a medical fluid system, e.g., dialysis, user interface, which is convenient for patients and caregivers to use. The user interface in one embodiment includes a video screen and a plurality of off-screen input devices, such as buttons or membrane switches and a rotary knob. In one implementation, the buttons or membrane switches include a stop button (e.g., red), back button (e.g., gray), a confirm button (e.g., green), a voice repeat/call center button (e.g., blue) and a rotary knob that interacts with each of the buttons to perform substantially all setup and therapy actions. The user interface can also include two hidden buttons, which are accessed by the nurse, practitioner or service person. The hidden buttons in one implementation put the medical fluid or dialysis instrument respectively in a clinician's mode (for certain settings the nurse/clinician, but not the patient, perform therapy actions) or a service mode (for access via service people only).

The visible buttons are each backlit in one embodiment. Processing and memory are programmed or configured in one embodiment to backlight a button only when it is active in context. Such an arrangement allows the user not to have to remember all of the functions of each button. Instead, the user only has to see which button is backlit. The buttons also have repeatable functionality, which combines to form an overall intuitive system that performs much of the “thinking” for the user. It is possible that multiple buttons are backlit at the same time. However, any button that is not backlit will not produce any effect at the present time, which eliminates that button from the set of buttons from which the user has to choose.

The hidden function buttons are not backlit in one embodiment. The hidden buttons can be felt underneath the outer layer of the user interface, which can be a thin plastic. In one embodiment, the nurse's or clinician's hidden button is located above the voice repeat/call center button, while the service button is located between the rotary knob and the voice repeat/call center button. In one implementation, each mode is entered by pressing and releasing the relevant hidden button twice followed by a press and release of the confirm key. This sequence of three distinct states is meant to prevent the patient from accidentally entering a mode by simply pressing one of the hidden keys inadvertently.

In one embodiment, the hidden buttons are only active during a startup of the system. The nurse/clinician or service person has to enter the clinician's or service mode during the startup sequence, and in one embodiment, within a certain time period of the startup sequence.

In an embodiment, a dialysis system can include a dialysis instrument having a pump actuator, a disposable dialysis fluid carrying set having a pump chamber operable with the pump actuator, and a graphical user interface (“GUI”). The GUI can include a selection highlighting input device and a selection entering input device. The selection highlighting input device can be configured to perform a screen navigation selection, a parameter selection including a parameter related to the pump actuator and the pump chamber, and a value selection including a value related to the pump actuator and the pump chamber.

In an embodiment, the selection entering input device includes a confirm button.

In another embodiment, the selection highlighting input device includes a rotary knob.

In an embodiment, the rotary knob includes a plurality of detents each corresponding to a different screen navigation selection, parameter selection, or value selection.

In a further embodiment, the screen navigation selection and associated entry causes the GUI to display a sub-screen from a current screen.

In an embodiment, the parameter selection and associated entry causes the GUI to display values or entries for a selected parameter.

In an embodiment, the value selection and associated entry causes the GUI to enter a value for a parameter.

In still another embodiment, the GUI includes a back input programmed to reverse a selection and associated entry of the screen navigation selection.

In an embodiment, the GUI includes a stop input programmed to stop an alarm posted in response to a selection and associated entry of the screen navigation selection.

In an embodiment, the GUI includes a back input programmed to display an informational screen during a treatment performed via the dialysis instrument.

In still a further embodiment, the GUI includes a stop input programmed to pause the pump actuator during a treatment performed via the dialysis instrument.

In an embodiment, the GUI includes a plurality of screen navigational selections, one of which is displayed only when available for selection.

In an embodiment, the GUI includes a plurality of parameter selections, one of which is displayed only when available for selection.

In yet another embodiment, the GUI includes a navigational input device. The GUI can also be configured to selectively light the selection highlighting input device, the selection entering input device, and the navigational input device when any of these input devices are available for input to the GUI.

In an embodiment, the GUI includes at least one hidden button and is programmed to use the hidden button to enter an instrument mode hidden from a patient using the instrument.

In yet a further embodiment, the GUI includes a display device and is configured to dim the display device after a period of inactivity.

In an embodiment, the GUI is configured to brighten the display device upon movement of the selection highlighting device.

In an alternate embodiment, a dialysis system can include a dialysis instrument having a pump actuator, a disposable dialysis fluid carrying set having a pump chamber operable with the pump actuator, and a graphical user interface (“GUI”). This GUI can include a navigational input device, a selection highlighting input device and a selection entering input device. Each input device can be configured to control a parameter related to the pump actuator and the pump chamber. The GUI can be programmed to selectively light the input devices only when any of these input devices are available for input into the GUI.

In an embodiment, the GUI includes a stop input device programmed to stop the pump actuator. The GUI can be programmed to selectively light the stop input device only when the stop input device is available for input into the GUI.

In still another embodiment, a dialysis system can include a dialysis instrument and a graphical user interface (“GUI”). The GUI can include a selection highlighting input device and a selection entering input device. The selection highlighting input device can be configured to allow a user to enter a screen navigation selection, a parameter selection, and a value selection without the use of a keyboard.

It is accordingly an advantage of the present disclosure to provide a user interface that is easy to use.

It is another advantage of the present disclosure to provide a user interface that is intuitive.

It is a further advantage of the present disclosure to provide a user interface that provides full operability of a dialysis instrument without the use of a keyboard.

Additional features and advantages are described herein, and will be apparent from, the following Detailed Description and the figures.

### BRIEF DESCRIPTION OF THE FIGURES

FIG. 1 is a perspective view of one embodiment of a dialysis system that employs the graphical user interface of the present disclosure.

FIG. 2 is a perspective view of one embodiment of a dialysis system and disposable cassette useable with the graphical user interface of the present disclosure.

FIG. 3 is an elevation view illustrating one embodiment of a graphical user interface (“GUI”) of the present disclosure.

FIG. 4 is an elevation view illustrating one embodiment of a program therapy settings power-up screen of the present disclosure.

FIG. 5 is an elevation view illustrating one embodiment of a clinician menu screen of the present disclosure.

FIG. 6 is an elevation view illustrating one embodiment of a clinician settings screen of the present disclosure.

FIG. 7 is an elevation view illustrating one embodiment of a clinician settings screen of the present disclosure with fill mode highlighted.

FIG. 8 is an elevation view illustrating one embodiment of a clinician settings screen of the present disclosure with low fill highlighted.

FIG. 9 is an elevation view illustrating one embodiment of a clinician settings screen of the present disclosure with minimum drain volume highlighted.

FIG. 10 is an elevation view illustrating one embodiment of a clinician settings screen of the present disclosure with heater bag empty highlighted.

FIG. 11 is an elevation view illustrating one embodiment of a clinician settings screen of the present disclosure with personal data key (“PDK”) highlighted.

FIG. 12 is an elevation view illustrating one embodiment of a confirm changes screen of the present disclosure.

FIG. 13 is an elevation view illustrating one embodiment of a clinician menu screen of the present disclosure with accept settings highlighted.

FIG. 14 is an elevation view illustrating one embodiment of a clinician menu screen of the present disclosure with therapy settings highlighted.

FIG. 15 is an elevation view illustrating one embodiment of a therapy settings screen of the present disclosure with therapy type highlighted.

FIG. 16 is an elevation view illustrating one embodiment of a therapy settings/therapy type edit screen of the present disclosure in a first state.

FIG. 17 is an elevation view illustrating one embodiment of a therapy settings/therapy type edit screen of the present disclosure in a second state.

FIG. 18 is an elevation view illustrating one embodiment of a therapy settings screen of the present disclosure with total therapy volume highlighted.

FIG. 19 is an elevation view illustrating one embodiment of a therapy settings screen of the present disclosure in a first state with number of day fills highlighted.

FIG. 20 is an elevation view illustrating one embodiment of a therapy settings screen of the present disclosure in a second state with number of day fills highlighted.

FIG. 21 is an elevation view illustrating one embodiment of a therapy settings screen of the present disclosure in a first state with day fill volume highlighted.

FIG. 22 is an elevation view illustrating one embodiment of a therapy settings screen of the present disclosure in a second state with day fill volume highlighted.

FIG. 23 is an elevation view illustrating one embodiment of a therapy settings screen of the present disclosure with night therapy time highlighted.

FIG. 24 is an elevation view illustrating one embodiment of a therapy settings screen of the present disclosure with night fill volume highlighted.

FIG. 25 is an elevation view illustrating one embodiment of a therapy settings screen of the present disclosure with last fill volume highlighted.

FIG. 26 is an elevation view illustrating one embodiment of a therapy settings/last fill volume edit screen of the present disclosure.

FIG. 27 is an elevation view illustrating one embodiment of a check initial drain settings alarm screen of the present disclosure.

FIG. 28 is an elevation view illustrating one embodiment of a check initial drain settings screen of the present disclosure.

FIG. 29 is an elevation view illustrating one embodiment of a check initial drain settings/initial drain volume edit screen of the present disclosure.

FIG. 30 is an elevation view illustrating one embodiment of a therapy settings screen of the present disclosure with last fill dextrose highlighted.

FIG. 31 is an elevation view illustrating one embodiment of a therapy- paused screen of the present disclosure.

FIG. 32 is an elevation view illustrating one embodiment of a therapy information/logs and settings screen of the present disclosure.

FIG. 33 is an elevation view illustrating one embodiment of a drain screen displayed during therapy via the graphical user interface of the present disclosure.

FIG. 34 is an elevation view illustrating one embodiment of a fill screen displayed during therapy via the graphical user interface of the present disclosure.

FIG. 35 is an elevation view illustrating one embodiment of a dwell screen displayed during therapy via the graphical user interface of the present disclosure.

FIG. 36 is an elevation view illustrating one embodiment of a therapy summary screen displayed during therapy via the graphical user interface of the present disclosure.

FIG. 37 is an elevation view illustrating one embodiment of an alarm screen displayed during therapy via the graphical user interface of the present disclosure.

FIG. 38 is an elevation view illustrating one embodiment of a screen displayed when the user wishes to call for help.

### DETAILED DESCRIPTION

### System Generally

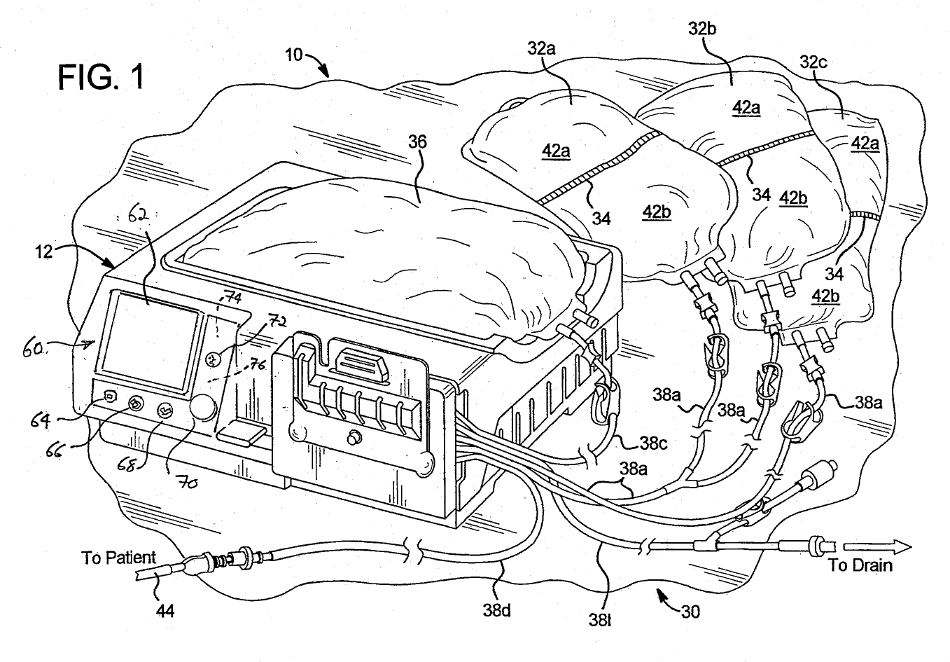

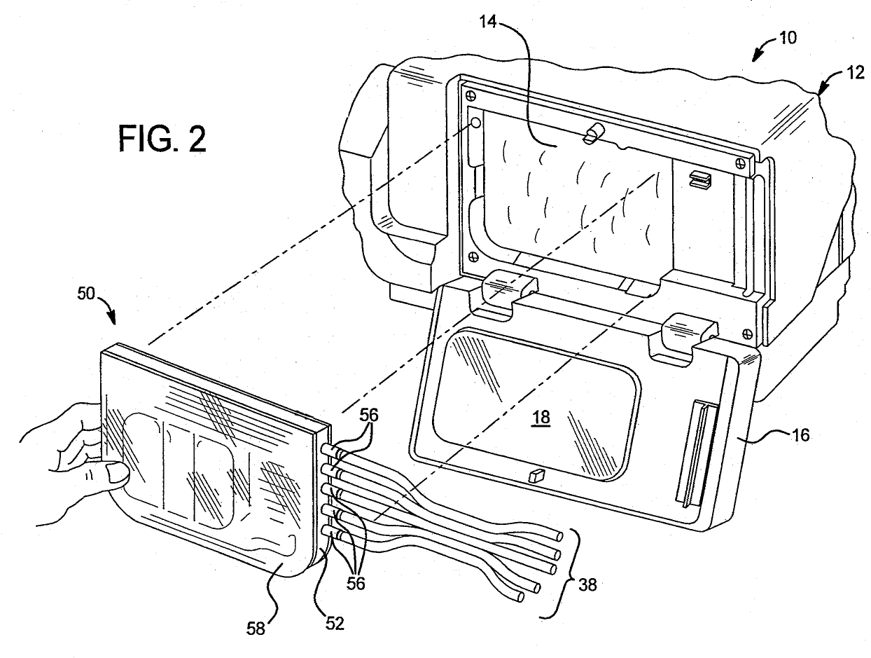

Referring now to the drawings and in particular to FIGS. 1 to 2, a renal failure therapy system 10 is provided. System 10 is applicable generally to any type of renal failure therapy system, such as peritoneal dialysis (‘PD”), hemodialysis (“HD”), hemofiltration (“HF”), hemodiafiltration (“HDF”) and continuous renal replacement therapy (“CRRT”). The valve configurations described below could also be used outside of the renal field, such as for medication delivery in general or for blood processing. For ease of illustration, however, system 10 is described in general as a dialysis system, and in one particularly well-suited application, as a PD system.

System 10 in the illustrated embodiment includes a dialysis instrument 12. Dialysis instrument 12 is configured for whichever type of renal failure therapy system is used. Dialysis instrument 12 includes a central processing unit (“CPU”) and a plurality of controllers (e.g., safety, valve, heater, pump, video, audio controllers) operable with the CPU. CPU operates with a graphical user-machine interface (“GUI”) 60 discussed in detail below, e.g., via the video controller. GUI 60 includes a video monitor 62 and a plurality of input devices discussed in detail below.

As seen in FIG. 1, dialysis instrument 12 accepts and operates with a disposable apparatus 30. Disposable apparatus 30 includes one or more supply bags 32a to 32c (referred to herein collectively as supply bags 32 or individually, generally as supply bag 32), shown here as dual-chamber supply bags separating two fluids via a peel or frangible seal 34. Disposable set 30 also includes a drain bag (not illustrated), a warmer bag 36, bag tubes 38a to 38d (referred to herein collectively as tubing or tubes 38 or individually, generally as tube 38) and a disposable pumping/valve cassette 50 (FIG. 2).

Depending on the type and structure of the renal failure therapy system 10, one or more of the items of disposable apparatus 30 may not be needed. For example, system 10 can pump spent fluid to a house drain, such as a toilet or sink, instead of a drain bag. System 10 can also include an inline heater, in which case warmer bag 36 is not needed.

While three supply bags 32 are shown, system 10 can employ any suitable number of supply bags. Supply bags 32 are shown having multiple chambers 42a and 42b, separated by frangible seal 34, and hold different solutions depending on the type of therapy employed. For example, chambers 42a and 42b can hold buffer and glucose for PD or acetate and bicarbonate solution for HD. Supply bags 32 are alternatively single chamber bags, which hold a single premixed solution, such as premixed PD or HD dialysate.

As seen in FIGS. 1 and 2, a disposable cassette 50 connects to supply bags 32, drain bag and warmer bag 36 via tubes 38a, 38b and 38c, respectively. Tube 38d runs from cassette 50 to a patient connection 44. Cassette 50 in one embodiment includes a rigid structure having rigid outer walls 52, a base wall from which inner pump chambers, valve chambers and inner fluid pathways extend, rigid fluid ports 56 that connect sealingly to tubing 38, and a pair of flexible membranes or sheets 58 sealed to outer rigid walls 52 and possibly to inner rigid walls as well.

Instrument 12 can actuate the pump and valve chambers of cassette 50 pneumatically, mechanically or both. The illustrated embodiment uses pneumatic actuation. The HomeChoice® APD system, which could operate or be made operable with cassette 50, uses a pneumatic system described in U.S. Pat. No. 4,826,482 (“The '482 Patent”), the entire contents of which are incorporated herein by reference. In the illustrated embodiment, instrument 12 includes a membrane 14, which creates different sealed areas with sheeting 58 at each of the pump and valve chambers of cassette 50. Membrane 14 moves with the sheeting 58 in those areas to either open/close a valve chamber or to pump fluid through a pump chamber. An interface plate is located behind membrane 14 and forms part of each of a pair of fixed volume pump chambers in combination with the pump chambers (actually pump chamber portion) of cassette 50 discussed below.

Instrument 12 in the illustrated embodiment includes a door 16, which closes against cassette 50. Door 16 includes a press plate 18, which can be operated mechanically (e.g., via the closing of the door) and/or pneumatically (e.g., via an inflatable bladder located in the door behind the press plate). Pressing plate 18 against cassette 50 in turn presses cassette 50 against a pumping membrane 14 that cooperates with the sheeting 58 of cassette 50 to pump fluid and open and close valves.

A cassette interface plate (not seen) is located behind membrane 14. The cassette interface plate is configured to apply positive or negative pressure to the coactive membrane 14 and cassette sheeting 58 at the different valve and pump areas. For example, positive pressure is applied to membrane 14/sheeting 58 at an area of the sheeting located within the internal walls of cassette 50 defining the pump chambers to push fluid out of the pump chambers. Negative pressure is applied to membrane 14/sheeting 58 at that same area to pull fluid into the pump chambers. Positive pressure is applied to membrane 14/sheeting 58 at an area of the sheeting within the internal walls of cassette 50 defining the valve chambers to close off an outlet port of the valve chambers. Negative pressure is applied to membrane 14/sheeting 58 at that same area of cassette 50 to open an outlet of the valve chambers.

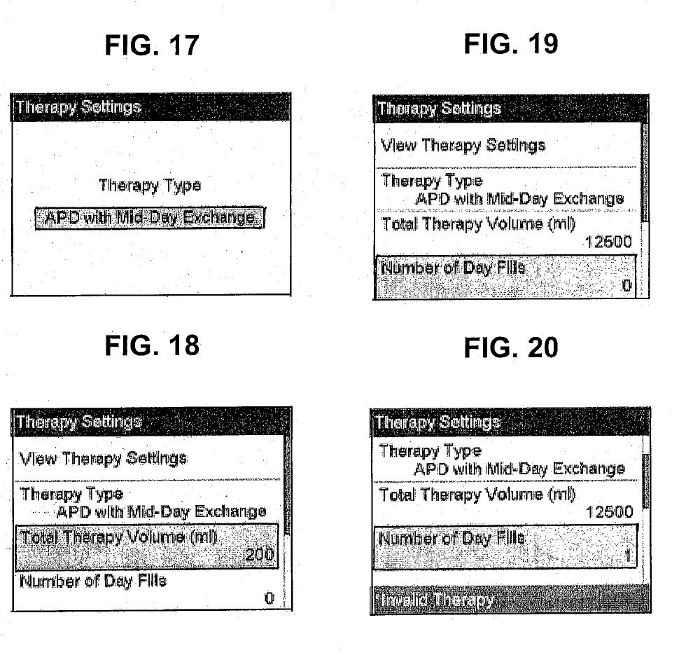

U.S. Pat. No. 6,814,547 (“the '547 patent”) discloses a pumping mechanism in connection with FIGS. 17A and 17B, incorporated herein by reference, which uses a combination of pneumatic and mechanical actuation. FIGS. 15, 16A and 16B of the '547 Patent, incorporated herein by reference, teach the use of mechanically actuated valves. The system could also be configured to operate with the pass-through valves of the present disclosure.

### Graphical User Interface Generally

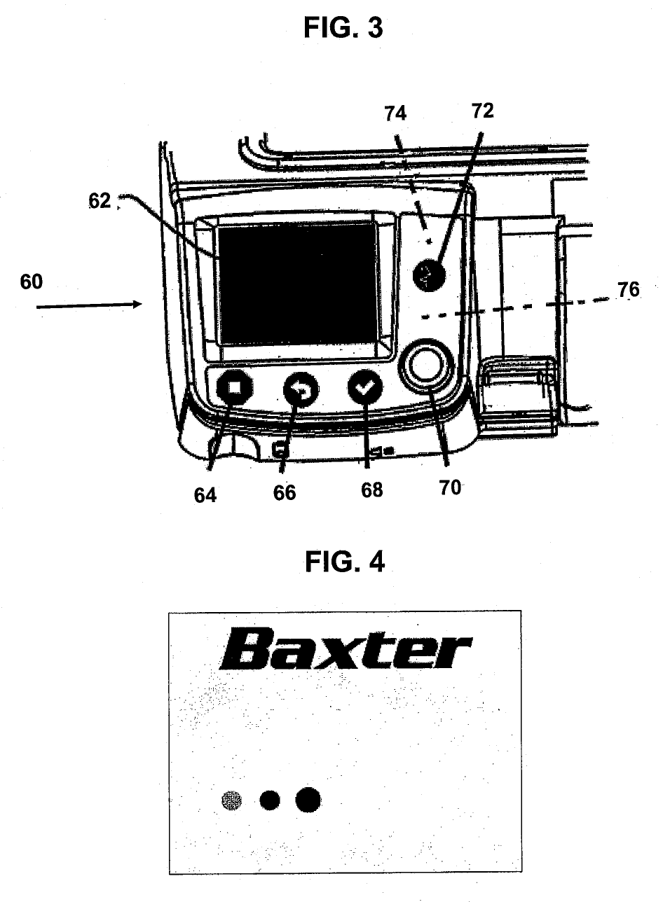

FIG. 3 illustrates one embodiment of GUI 60, which includes video monitor 62 and a plurality of input devices including a stop button 64, a back button 66, a confirm button 68, a rotary knob 70, and a voice repeat/call center button 72. In one implementation, stop button 64 is backlit red when active, back button 66 is backlit gray when active, confirm button 68 is backlit green when active, rotary knob 70 is backlit blue when active, and voice repeat/call center button 72 is backlit blue when activated.

Voice guidance is an option that the patient either turns on or off. If voice guidance is activated, the voice command associated with the present screen is automatically recited. For example, in this activated mode, the voice repeat/call center button 72, when pressed, repeats the last voice command during therapy. If however, voice guidance is deactivated but the patient or caregiver selects voice repeat/call center button 72 while the button is backlit, the voice guidance system enunciates whatever text appears on the screen.

GUI 60 also includes a clinician mode hidden button 74 and a service mode hidden button 76 (element numbers mark where hidden buttons are located behind outer skin of front panel). In one embodiment, the hidden buttons are only active during a startup of the system. When the machine powers-up, the video monitor shows an initialization screen as seen in FIG. 4, e.g., screen with moving items indicating that the system is waiting for initialization process to complete. A certain time frame, e.g., five seconds, is provided in which the nurse/clinician or service person can touch and release one of the hidden buttons 74 or 76. At that point, the confirm button is backlit and active. If the nurse/clinician or service person touches and releases the confirm button within the allotted time, the machine enters the clinicians mode or the service mode, depending upon which button is selected.

The clinician's mode selected via hidden button 74 allows the nurses or clinicians to make therapy changes that the patient should not make, such as, choosing between an adult mode or pediatric mode. The machine provides limited functionality to the patient and full medical functionality to the nurse or clinician.

Once in clinician's mode, the nurse or clinician is provided with a scrollable list of options from which to select. The nurse or clinician uses rotary knob 70 to scroll through the list of options. Rotary knob 70 at this point is backlit to let the nurse or clinician know that the knob is active and can be turned to scroll through the list of options to select one of the options.

A selection of one of the options from the main list of options can cause video monitor 62 to display a sub-menu of options specific to the selected option from the main menu. Alternatively or perhaps after the selection of an option from the sub- menu, video monitor 62 displays a list of selectable parameters.

For example, the main menu in the clinician's mode allows the clinician to scroll through the main menu option to select a change therapy option. The clinician rotates knob 70 until display device 62 highlights the change therapy option. At that point, the clinician selects confirm key 68. Display device 62 now shows the therapy menu. Display device 62 highlights the first selection in the therapy menu (one of the selections lit at all times in one embodiment). Knob 70 is backlit and active. The clinician rotates knob 70 again to highlight and select a desired parameter to change, e.g., total therapy volume. The clinician selects confirm key 68 again. Display device 62 now shows the total therapy volume screen, which includes a plurality of selectable total therapy volumes, as discussed in detail below. Display device 62 highlights the first fill volume. Knob 70 is again backlit and active but its function has changed from a parameter selector (e.g., total therapy volume) to a value selector (e.g., incrementing from 200 ml to 80,000 ml by varying ml increments as shown below).

Knob 70 can highlight other types of selections, such as “yes” versus “no.” Confirm key 68 is used to enter one of “yes” or “no.” Knob 70 can highlight languages, e.g., English, Spanish, French or Italian. Confirm key 68 is used to enter one of the languages for both written text and voice guidance. These examples show that knob 70 can scroll though values or any of two or more choices. Thus, it should be appreciated that rotary knob 70 enables the user to perform each of: (i) screen navigation; (ii) parameter selection including at least one parameter related to the pump actuator and pump chamber; and (iii) value selection including at least one value related to the pump actuator and pump chamber.

Rotary knob 70 in one embodiment can be rotated freely as much as the user wants to rotate the knob. That is, the knob does not have a physical hard stop in one embodiment. When in a screen navigational mode, the screen selections wrap around the navigational selections. That is, the user continues to rotate knob 70 in the same direction so that the highlight passes the last (or first) selection and returns to the top (or bottom) of display device 62 to highlight the first (or last) selection. Alternatively, the highlight moves from left to right or right to left.

Highlighting amongst selections, such as “yes” or “no” or language selections operate the same as with navigational selections in one embodiment. Turning knob 70 in either direction causes the highlighter to toggle between “yes” or “no” or, e.g., “English,” “Spanish,” “French,” or “Italian.” Alternatively, 180 degrees of a full 360 degree turn in either direction is dedicated to “yes.” The other 180 degrees of the full 360 degree turn in either direction is dedicated to “no.” Or, a first 120 degrees of a full 360 degree turn in either direction is dedicated to “English.” The second 120 degrees of the full 360 degree turn in either direction is dedicated to “Spanish.” The third 120 degrees of the full 360 degree turn in either direction is dedicated to “French.” As more languages are added, the range of degrees dedicated is reduced proportionally.

In one embodiment, soft-stops are provided for values. For example, if the total therapy volume for a particular type of therapy has a range of 200 ml to 80,000 ml of dialysis solution, turning knob 70 counterclockwise moves the highlight bar at set intervals so that the highlighted fill volume decreases. When the lowest possible fill volume, e.g., 200 ml, is reached, turning rotary knob 70 further in the counterclockwise direction has no effect. Turning knob 70 clockwise moves the highlight bar at set intervals so that the highlighted fill volume increases. When the highest possible fill volume, e.g., 80,000 ml, is reached, turning rotary knob 70 further in the clockwise direction has no effect. In another embodiment, clockwise movement of knob 70 is dedicated to “yes” while counterclockwise movement of knob 70 is dedicated to “no.” In a further embodiment, soft-stops are provided for language selections or for “yes” or “no” selections as well. For example, turning knob 70 counterclockwise moves the highlight bar to the first language, e.g. English, while turning knob 70 clockwise moves the highlight bar to the last language, e.g. Italian.

In an alternative embodiment, if either the lowest or highest possible values are reached and the knob 70 is turned counterclockwise or clockwise, respectfully, the value wraps around to the highest or lowest value, respectfully. For example, when the lowest possible therapy volume, e.g., 200 ml, is reached, turning rotary knob 70 further in the counterclockwise direction wraps the value around to the highest value, e.g., 80,000 ml, decreasing from that highest value as the knob is further turned in the counterclockwise direction. In another embodiment, when the first language is reached, e.g. English, turning knob 70 further in the counterclockwise direction wraps the language around to the last language, e.g. Italian, continuing through the list of languages in reverse order. When the highest possible therapy volume, e.g., 80,000 ml, is reached, turning rotary knob 70 further in the clockwise direction wraps the value around to the lowest value, e.g., 200 ml, increasing from that lowest value as the knob is further turned in the clockwise direction. In another embodiment, when the last language is reached, e.g. Italian, turning knob 70 further in the clockwise direction wraps the language around to the first language, e.g. English, continuing through the list of languages in forward order.

If it is desired to change an entered or confirmed selection, the user can repeat the sequence, making a different selection. For example, if the user enters a first language from the language selection screen, the display device 62 in one embodiment next displays the screen that allowed the user to enter the language selection screen. This way, the user can again select the language selection option, move to the language selection screen, select a new language and then move again to the next highest screen. This sequence can be repeated as many times as necessary.

Values can be re-entered similarly. For example, if the user enters a first value from the total therapy volume selection screen, the display device 62 in one embodiment next displays the screen that allowed the user to enter the fill volume selection screen. In this regard, the user can again select the fill volume selection option, move to the total therapy volume screen, select a new fill volume and then move again to the next highest screen. This sequence too can be repeated as many times as necessary. In one embodiment, values cannot be changed after therapy has begun.

In one embodiment, once the user navigates to a particular screen of GUI 60, GUI 60 continues to show the screen until the user navigates off of the screen. In another embodiment, the screen times out after a period of time and returns to a home screen.

In either case, GUI 60 can display a screensaver. For example, GUI 60 can be configured to dim display device 62 after a certain period of inactivity, e.g., ten minutes. Here, GUI 60 turns off instrument 12 backlights, active buttons, and even buttons that would otherwise be active. However, the backlight of rotary knob 70 remains a faint, pale blue in one embodiment that is non-intrusive to the patient. Turning knob 70 one way or another causes the buttons and display to be re-lit and active and the buttons that should be active to become active. Only the buttons that are active will be backlit. Knob 70 provides a large target for the patient to intuitively notice, even when the patient may have just been asleep. A patient who has been asleep may hit the stop button inadvertently, causing the machine to stop pumping when it is not desirable to do so. The two-stage rotary knob 70 turn followed by the hitting of the button tends to prevent such a inadvertent entry. System 10 remains active throughout. Turning rotary knob 70 brings display device 62 back to an active state.

Hitting stop button 64 or back button 66 in the active or fluid moving state causes GUI 60 to enter a navigational mode as described in detail below. Here, knob 70 returns to its selection highlighting operating state. Pressing the stop button 64 during active therapy will cause fluid flow to stop and will cause a list of user options to be displayed on display device 62, as shown in FIG. 31.

### Example Screens for Clinician Settings

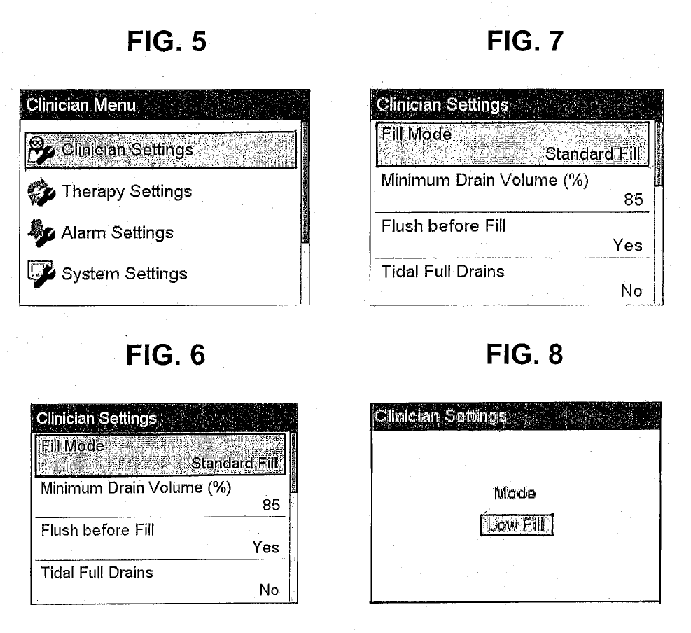

Back button 66 allows the user to navigate backwards from one screen to another, e.g., during therapy. During setup or menu selection, back button 66 moves the user back or up a level to the previous screen without entering a selection. For example, as seen in FIG. 5, if the clinician is at the main menu and selects (i.e. highlights and confirms) “therapy settings”, e.g., from “clinician settings,” “therapy settings,” “alarm settings” and “system settings,” GUI 60 shifts display 62 to the “adjust therapy parameters” screen, which includes for example ten selections. The clinician then highlights “fill volume” and presses confirm button 68. If the clinician at that point decides not to change the fill volume, the patient presses back button 66, which pulls GUI 60 back to the “adjust therapy parameters.” No change in fill volume is entered, even if the clinician had rotated knob 70 (but not hit confirm button 68). Again, back button 66 is backlit (e.g., gray) whenever it is active and not backlit when not active, e.g., at the main menu at which there is no prior screen.

Thus at pre- and post-therapy (setup and tear down), back button 66 operates as a navigational tool, e.g., for certain screens thereof. During set-up, certain screens of display device 62 are appropriate for the back button 66, while others are not. For example, if GUI 60 provides two screens telling the patient to gather supplies, back button 66 is backlit and active during the two screens because no action is taken in gathering supplies that cannot be undone. If the patient wishes to go back and check that the proper supplies have been gathered, back button 66 allows the patient to do this during those two screens and perhaps even during one or more screens following the “gather supplies” screens.

Other points in pre- and post-therapy are not reversible, so back button 66 is not backlit or active. For example, once a supply bag 32 is pierced or a frangible seal is broken, the action cannot be reversed. Therefore, back button 66 is not backlit or active.

During active therapy, fluid is being moved. Here, back button 66 is active and backlit. During active treatment, back button 66 causes GUI 60 to display a “logs and settings” menu. “Logs and settings” allows the user to view settings for the current therapy parameters, how much ultrafiltration (“UF”) has been removed from the patient, how much fresh fluid has been delivered to the patient, how much spent fluid has been removed from the patient, current dwell time, etc.

“Logs and settings” also allows the user to change the lighting or brightness for display device 62, enable/disable voice guidance, change the loudness of voice guidance or other parameters that are considered passive. Therapy related parameters, such as pump volume or pump speed, cannot be changed during active therapy in one embodiment.

Stop button 64 is active and backlit (e.g., red) during active therapy when fluid is moving. When the patient or clinician presses stop button 64 during active therapy, instrument 12 shuts down all pumping so that fluid movement stops. A patient dwell phase is considered part of active therapy. Pressing stop button 64 during this phase will have the same result as pressing stop button 64 during a fill or drain phase. As there may be fluid flowing from supply bag 32 to heater bag 36 during dwell, pressing stop button 64 will halt fluid flow and progression of therapy.

As seen in FIGS. 5 and FIG. 6, using knob 70 to select “clinician settings” from “clinician main menu” causes the “clinician settings screen” to appear as seen in FIG. 6. In one embodiment, the clinician rotates knob 70 in “fill mode” to select either a “standard fill” or a “low fill.” The “clinician settings screen” in the illustrated embodiment for a standard fill allows the user to turn knob 70 to select one of a “fill mode” sub-menu, “minimum drain volume” sub-menu, “flush before fill” sub-menu, and “tidal full drains” sub-menu. The clinician rotates knob 70 to increment “minimum drain volume” in increments of five %. The clinician rotates knob 70 to select between “yes” and “no” for each of “flush before fill,” “tidal full drains,” “adjust dwell up,” “adjust dwell down,” and “heater bag empty.” The “clinician settings” also allows the user to select a language as discussed, lock the settings, and enable a personal data key.

After selecting “low fill” for a “fill mode” using knob 70, “low fill” is highlighted as seen in FIG. 8. A low fill mode sub-screen is displayed on display device 62, which allows the clinician to select and enter “minimum drain time.” The “minimum drain time” in one embodiment has a range of one to thirty minutes, and a default set to zero minutes, so that a number must be entered. Again, “minimum drain time” in one embodiment is needed only for low fills.

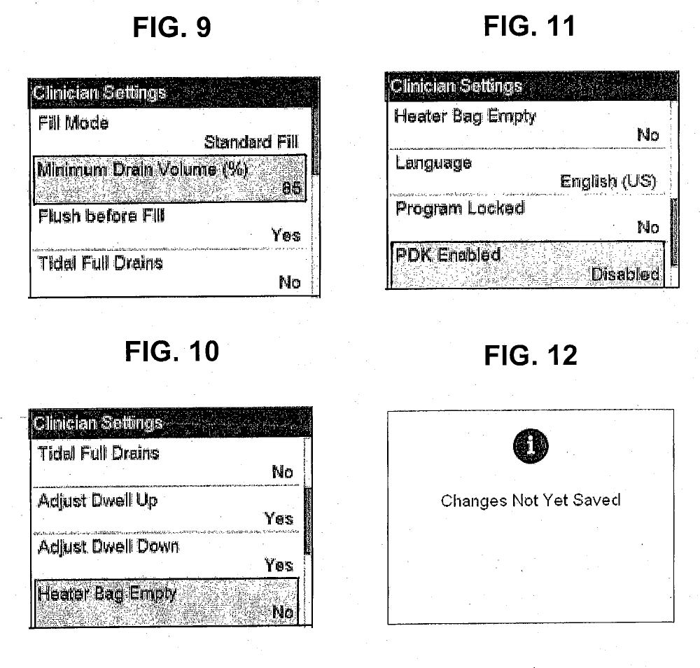

Referring to FIG. 9, the “minimum drain volume” percentage selected for either standard or low mode sets the minimum limit for draining the patient. If actual volume drained is less than the selected percentage of a volume delivered during fill, system 10 alarms that there is a low drain volume. If actual volume drained is more than the selected percentage of the volume delivered during fill, system 10 alarms and assumes that the patient has been drained fully and proceeds to the next fill.

The “flush before fill” option allows the system to use a certain volume of fresh fluid to flush the lines during priming. The flush volume is then pumped to drain. The “tidal full drains” option (standard fill mode only) allows for a tidal therapy to be performed in which intermediate drains and fills are partial. The “adjust dwell up” and “adjust dwell down” options allow the system to adjust dwell times in case the patient drains/fills more or less quickly than expected to keep end therapy time constant.

The “heater bag empty” option selected as seen in FIG. 10 allows system 10 to begin therapy with heater bag 36 (FIG. 1) empty. Locking the program allows the patient to view but not to change the therapy settings. The personal data key selected as seen in FIG. 11 stores therapy data for the patient, e.g., for diagnostics.

A “negative UF limit” option and “positive UF limit” option are provided in one embodiment when the “low fill” option is selected. These options set a percentage for the drain volume under or over, respectively, the fill volume. If the percentage is below “negative UF limit” or above “positive UF limit,” system 10 alarms.

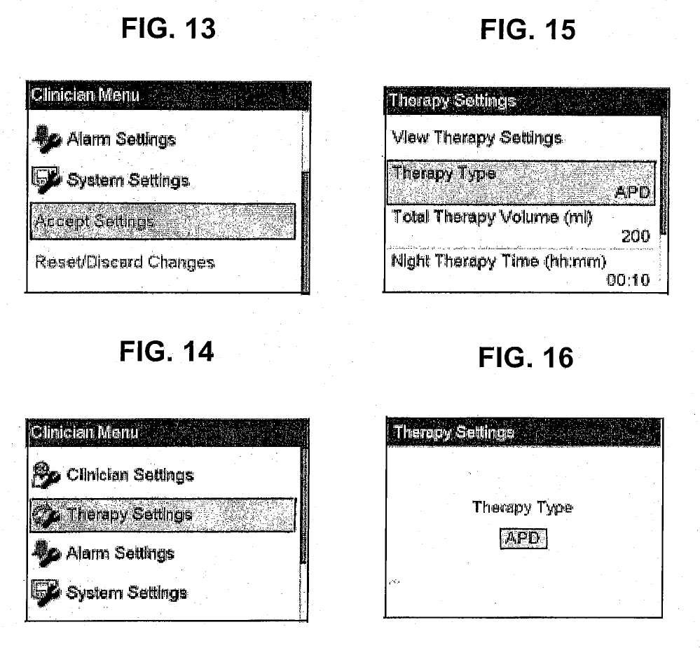

In an alternative embodiment, once all the above disclosed settings are chosen, the patient selects back button 66, which returns the clinician to a “changes not yet saved” screen as seen in FIG. 12. The clinician selects confirm button 68 to return the clinician to the “clinician menu” screen of FIG. 5. Here, the patient turns knob 70 to highlight “accept settings” as seen in FIG. 13. The other option in FIG. 13 is to “reset/disregard changes.” If no changes have been made via the clinician settings option, “accept settings” and “reset/disregard changes” options are grayed and non-active.

### Example Screens for Therapy Settings

FIG. 5 shows that the “clinician menu” also offers “therapy settings,” “alarm settings” and “system settings” for the clinician. In one embodiment, GUI 60 allows the patient to also make these setting changes (albeit some features may be disabled for the patient). In one embodiment, “therapy settings” allows the following parameters to be set: (i) therapy type including APD, APD with mid-day exchange, tidal and tidal with mid-day exchange; (ii) total therapy volume; (iii) night therapy time; (iv) night fill volume; (v) last fill volume; (vi) last fill dextrose; (vii) number of day fills (mid-day exchange therapies only); (viii) day fill volume (mid-day exchange therapies only); (ix) night tidal volume (tidal therapies only); (x) night expected UF (tidal therapies only); and (xi) full drains every (tidal therapies only).

In one embodiment, “alarm settings” allows the following parameters to be set: (i) last manual drain; (ii) last manual drain UF target (ml); (iii) last manual drain UF alarm; (iv) initial drain time (hh:mm) (low fill mode only); and (v) initial drain volume (ml).

In one embodiment, “system settings” allows the following parameters to be set: (i) loudness level; (ii) brightness level; (iii) auto dim enabled; (iv) voice guidance enabled; (v) therapy complete notice; (vi) comfort control (° C.); (vii) time format; (viii) time; (ix) date; (x) decimal separator; and (xi) system information.

FIG. 14 shows that the user has rotated knob 70 to highlight “therapy settings.” After pressing confirm button 68, display device 62 shows the “therapy settings” in FIG. 15. In FIG. 15, the user rotates knob 70 to highlight “therapy type” and presses confirm button 68, which causes a “therapy type edit” screen to appear in FIG. 16. In the illustrated embodiment, “therapy type edit” screen does not display all options at once. FIG. 16 highlights a default automated peritoneal dialysis (“APD”) selection. Turning knob 70 causes the highlighted display area to change the selection from “APD” to “APD with mid-day exchange” as seen in FIG. 17. The user presses confirm button 68 to cause the machine to run an APD with mid-day exchange therapy type rather than a standard APD therapy type. In general, mid-day exchange therapy adds an additional exchange during the day to an APD or tidal night therapy to improve treatment.

In FIG. 18, the user rotates knob 70 in the “therapy settings” screen to highlight “total therapy volume” and presses confirm button 68, which causes a total therapy volume edit screen to appear. Here, as discussed above, the user turns rotary knob 70 clockwise beginning at low end default of 200 ml. Fill volume increases by fifty ml increments for e.g., the first 2000 ml, 100 ml increments until reaching 5000 ml, and then increase by 500 ml increments until reaching a maximum of 80,000 ml.

In FIG. 19, the user rotates knob 70 in the “therapy settings” screen to highlight “number of day fills” and presses confirm button 68, which causes a number of day fills edit screen to appear. Here, as discussed above, the user turns rotary knob 70 clockwise beginning at low end default of zero to a desired number, e.g., one, and presses confirm 68. FIG. 20 shows that the number of day fills has increased to one but that an invalid therapy has been selected.

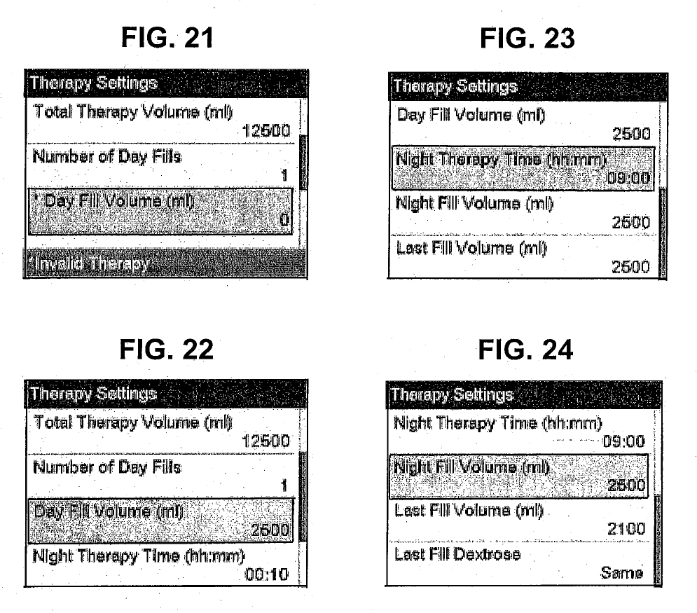

FIG. 21 shows that the user rotates knob until finding the parameter displayed with an asterisk, which in the example is the “day fill volume.” The user presses confirm 68 to enter a day fill volume edit screen. The user rotates knob 70 to change the day fill volume to a desired number, e.g., 2500 ml. The user presses confirm button 68 to enter the value. Display device 62 returns to the “therapy settings” screen as seen in FIG. 22, showing the day fill volume updated to 2500 ml and a valid therapy has been selected.

In FIG. 24, the user rotates knob 70 in the “therapy settings” screen to highlight “night fill volume (ml)” and presses confirm button 68, which causes a night fill volume edit screen to appear. Here, the user turns rotary knob 70 clockwise beginning at, e.g., zero ml, to a desired night fill volume, e.g., 2500 ml, and presses confirm 68 to set the night fill volume and return to the “therapy settings” screen.

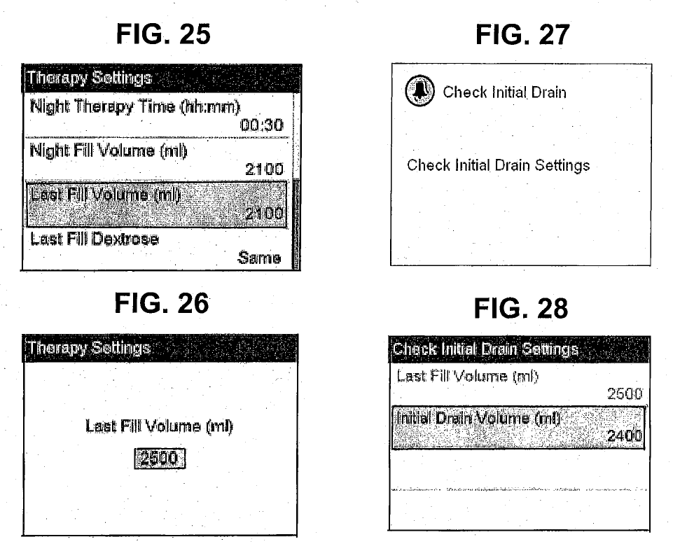

In FIG. 25, the user rotates knob 70 in the “therapy settings” screen to highlight “last fill volume (ml)” and presses confirm button 68, which causes a last fill volume edit screen to appear as seen in FIG. 26. Here, the user turns rotary knob 70 clockwise beginning at, e.g., 2100 ml to a desired last fill volume, e.g., 2500 ml, and presses confirm 68 to set the last fill volume and return to the “therapy settings” screen.

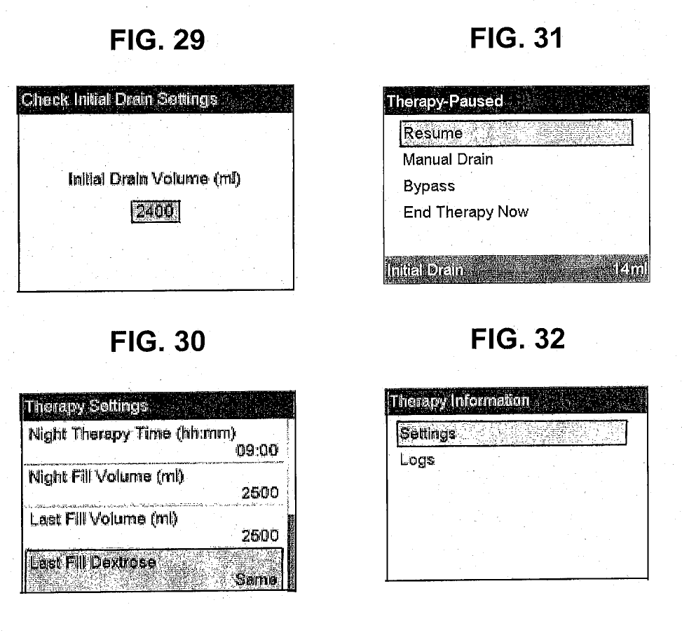

FIG. 27 illustrates that GUI 60 in one embodiment is configured to automatically alarm in certain conditions. In the instant case, whenever last fill volume is changed, GUI 60 alarms the user to check the initial drain volume. Here, the patient presses stop button 64 to clear the alarm. GUI 60 displays a “check initial drain settings screen” as seen in FIG. 28, which shows the last fill volume that has just been set (e.g., 2500 ml) and a previously set initial drain volume (e.g., 2400 ml). “Initial drain volume (ml)” is automatically highlighted and the patient presses confirm button 68, which causes an initial drain edit screen to appear as seen in FIG. 29. Here, the user turns rotary knob 70 clockwise beginning at, e.g., 2400 ml, to a desired initial drain volume, e.g., 2500 ml, and presses confirm 68 to set the initial drain volume (equal to the last fill volume) and return to the “check initial drain settings” of FIG. 28 (but showing volumes equal).

When in a screen, such as the “check initial drain settings” of FIG. 28, and no action is necessary, e.g., no need to select “initial drain volume” (last fill set above, initial drain set to match it), the user presses back button 66 to back up a level, here to the “therapy settings” screen as seen in FIG. 30. FIG. 30 shows another aspect of the GUI of the present disclosure, namely, that selections not always applicable can be added when applicable. In FIG. 30, a “last fill dextrose” selection is displayed and highlighted automatically, which is now applicable because last fill volume has been changed from zero to a positive volume. The “last fill dextrose” selection allows the last fill to be performed using dialysis fluid having the same dextrose level or a different dextrose level than the other night fills.

When all therapy parameters have been set, the patient presses back button 66 to back up one level. First, however, the user confirms all therapy changes made using the “changes not yet saved” screen of FIG. 12 and “clinician menu” modified to have “accept settings” of FIG. 13. User presses confirm button 68 when “accept settings” of FIG. 13 is highlighted to accept all changed clinician, therapy, alarm, and system settings.

### Example Screens Displayed during Therapy

FIG. 31 shows a “therapy-paused” screen that is displayed when stop button 64 is pressed during therapy. Pumping stops as discussed above. A status information line at the bottom of the “therapy-paused” screen shows the current cycle, e.g., initial drain and status of the cycle, e.g., 14 ml currently drained. The “resume” selection resumes therapy, e.g., re-starts fluid pumping. The “manual drain” option allows the patient to drain himself or herself manually with the pump stopped and thereafter disconnect from the system. The “bypass” selection will allow a current phase or cycle to be bypassed if allowed. The patient may be allowed to disconnect during dwell in which case a “disconnect” selection is displayed. In one embodiment, the “therapy-paused” screen shows only the options or selections eligible or available at the particular time in the treatment when stop button 64 is pressed.

In one embodiment, pausing therapy for an extended period of time, e.g., thirty minutes causes GUI 60 to sound and/or display an alarm reminding the patient to continue therapy or take other action. In one embodiment, a similar alarm occurs when an alarm has been muted for an extended period of time, e.g., ten minutes. In a power down situation in one embodiment, if therapy has not been stopped and power resumes within thirty minutes, therapy continues without alarm. If power resumes after thirty minutes but under two hours and therapy has not been stopped or if power resumes under two hours when the machine has been stopped, the system alarms. Any power failure lasting more than two hours causes the machine to abort therapy.

FIG. 32 shows a “therapy information” screen that is displayed when return button 66 is pressed during therapy. Pumping does not stop, instead, “settings” and “logs” selections are made available during treatment. As discussed above, system settings, e.g., lighting can be changed, while therapy settings can only be viewed in one embodiment.

### Example Therapy/Call Center Screens

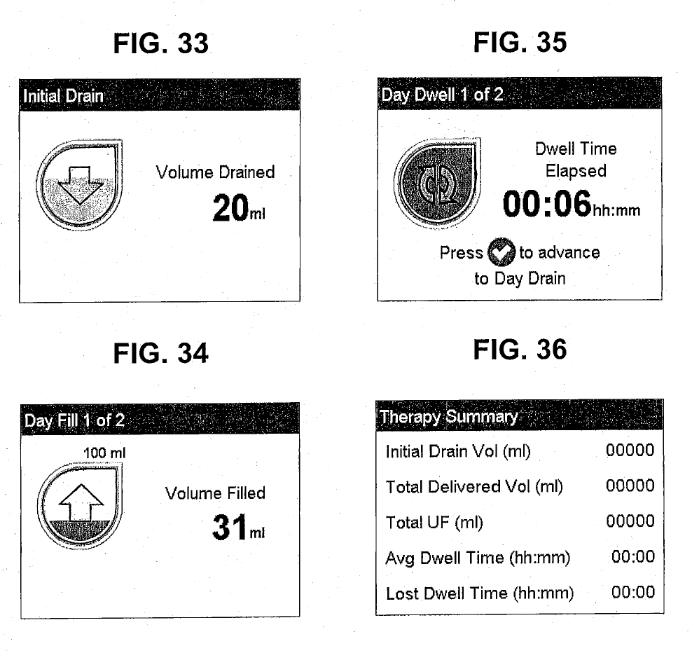

Referring now to FIG. 33, one example of a drain screen displayed on display device 62 of GUI 60 during therapy is illustrated. The drain screen is shown for an initial drain, however, a similar screen can be shown for any of the drain cycles. The drain screen shows a symbol with a downwardly pointing arrow indicating that the system is draining the patient. The amount currently drained is also shown numerically.

Referring now to FIG. 34, one example of a fill screen displayed on display device 62 of GUI 60 during therapy is illustrated. The fill screen is shown for a first day fill, however, a similar screen can be shown for any of the fill cycles. The fill screen shows a symbol with an upwardly pointing arrow indicating that the system is filling the patient. The amount currently filled is also shown numerically.

Referring now to FIG. 35, one example of a dwell screen displayed on display device 62 of GUI 60 during therapy is illustrated. The dwell screen is shown for a first day dwell, however, a similar screen can be shown for any of the dwell cycles. The dwell screen shows a symbol with circulating arrows indicating that the system is currently in a dwell state. The amount of time elapsed during the current dwell is also shown. The screen also informs the user that confirm button 68 can be pressed to cause the instrument 12 to stop the current dwell and advance to the next drain cycle.

Referring now to FIG. 36, one example of a therapy summary screen displayed on display device 62 of GUI 60 at the end of therapy is illustrated. The therapy summary screen shows the user or patient, for example, the initial drain volume, total volume of fresh solution delivered to the patient, total ultrafiltration removed from the patient, average time for all the dwells and any dwell time lost during treatment.

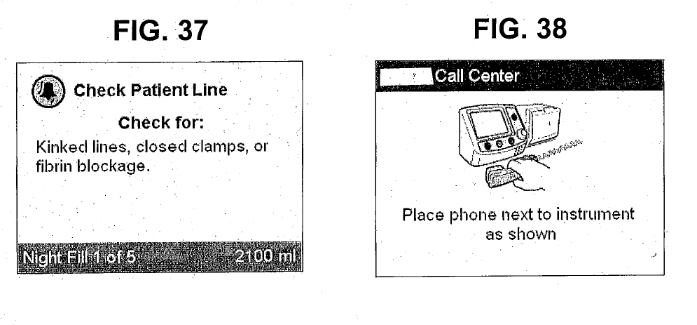

Referring now to FIG. 37, one example of an alarm screen displayed on display device 62 of GUI 60 during therapy is illustrated. The alarm screen illustrated is for a blocked patient line. GUI 60 includes other alarm screens. The screen of FIG. 37 shows a symbol of an alarm bell indicating an alarm or alert state. GUI 60 can additionally provide an auditory alert. The bottom of the screen shows that the alarm is occurring during a first fill. The alarm screen also provides a likely solution to the alarm condition, here to look for a kink in the patient line, a clamp closed on the patient line or for fibrin blockage within the patient line.

Referring now to FIG. 38, one example of a screen displayed on display device 62 after the user presses voice repeat/call center button 72 is illustrated. The voice repeat/call center screen informs the patient to place the patient's phone next to instrument 12, so that the speaker of instrument 12 can speak into the microphone of the telephone. In this manner, instrument 12 can provide instructions to the call center person to aid in diagnosing whatever problem the instrument may be experiencing.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A dialysis system comprising:
a dialysis instrument including a pump actuator;
a disposable dialysis fluid carrying set including a pump chamber operable with the pump actuator; and
a graphical user interface (“GUI”) including a selection highlighting input device and a selection entering input device, the selection highlighting input device configured to enable a user to perform each of: (i) screen navigation selection; (ii) parameter selection including at least one parameter related to the pump actuator and pump chamber; and (iii) value selection including at least one value related to the pump actuator and pump chamber.

2. The dialysis system of claim 1, the selection entering input device including a confirm button.

3. The dialysis system of claim 1, the selection highlighting input device including a rotary knob.

4. The dialysis system of claim 3, the rotary knob including a plurality of detents each corresponding to a different screen navigation selection, parameter selection or a value selection.

5. The dialysis system of claim 1, the screen navigation selection and associated entry via the selection entering input device causing the GUI to display a sub-screen from a current screen.

6. The dialysis system of claim 1, the parameter selection and associated entry via the selection entering input device causing the GUI to display values or entries for a selected parameter.

7. The dialysis system of claim 1, the value selection and associated entry via the selection entering input device causing the GUI to enter a value for a parameter.

8. The dialysis system of claim 1, the GUI further including a back input programmed to reverse a selection and associated entry of the screen navigation selection.

9. The dialysis system of claim 1, the GUI further including a stop input programmed to stop an alarm posted in response to a selection and associated entry of the screen navigation selection.

10. The dialysis system of claim 1, the GUI further including a back input programmed to display an informational screen during a treatment performed via the dialysis instrument.

11. The dialysis system of claim 1, the GUI further including a stop input programmed to pause the pump actuator during a treatment performed via the dialysis instrument.

12. The dialysis system of claim 1, the GUI further including a plurality of screen navigational selections, at least one of the screen navigational selections displayed only when available for selection.

13. The dialysis system of claim 1, the GUI further including a plurality of parameter selections, at least one of the parameter selections displayed only when available for selection.

14. The dialysis system of claim 1, the GUI (i) including a navigational input device and (ii) configured to selectively light the selection highlighting input device, the selection entering input device and the navigational input device when any of such input devices are available for input to the GUI.

15. The dialysis system of claim 1, the GUI including at least one hidden button, the GUI programmed to use the hidden button to enter a mode of the instrument hidden from a patient using the instrument.

16. The dialysis system of claim 1, the GUI including a display device and configured to dim the display device after a period of inactivity.

17. The dialysis system of claim 16, the GUI further configured to un-dim the display device upon a movement of the selection highlighting device.

18. A dialysis system comprising:
a dialysis instrument including a pump actuator;
a disposable dialysis fluid carrying set including a pump chamber operable with the pump actuator; and
a graphical user interface (“GUI”) including a navigational input device, a selection highlighting input device and a selection entering input device, each input device configured to control a parameter related to the pump actuator and pump chamber, the GUI programmed to selectively light the selection highlighting input device, the selection entering input device and the navigational input device only when any of such input devices are available for input into the GUI.

19. The dialysis system of claim 18, the GUI further including a stop input device programmed to stop the pump actuator, the GUI programmed to selectively light the stop input device only when the stop input device is available for input into the GUI.

20. A dialysis system comprising:
a dialysis instrument; and
a graphical user interface (“GUI”) including a selection highlighting input device and a selection entering input device, the selection highlighting input device configured to enable a user to select and enter each of: (i) a screen navigation selection; (ii) a parameter selection; and (iii) a value selection without the use of a keyboard.
